(19) United States
(12) Patent Application Publication
(10) Pub. No.: US 2016/0089272 A1
(43) Pub. Date: Mar. 31, 2016

(54) METHODS AND APPARATUS FOR PARTIAL OBSTRUCTION OF VISION FOR TREATING FORWARD HEAD POSTURE AND RELATED CONDITIONS

(71) Applicants: Conghua Li, Markham (CA); Allan Austin, Toronto (CA)

(72) Inventors: Conghua Li, Markham (CA); Allan Austin, Toronto (CA)

(21) Appl. No.: 13/745,555

(22) Filed: Jan. 18, 2013

(51) Int. Cl. A61F 9/04

(52) U.S. Cl. CPC A61F 9/04

(57) ABSTRACT

The invention provides an eyeglass-like apparatus and methods that may be used similarly to treat existing postural issues especially FHP and to prevent them in unaffected people. The apparatus may comprise a means for creating an obstruction in a zone within the field of vision of a person which size and position can be adjusted. The obstruction can be mounted in or on a lens, or a pair of lenses, or to a wearable frame without any lenses, or with non-vision-corrective translucent or transparent panes instead of lenses, or may be a small nose-mounted fixture, or may comprise a solid material, an electronic panel, or may be integrated as a component aspect of the shape and manufacture of a lens itself, or may be permanently fixed element in the lens, such as an etching, or it may be removable.

This application is a non-provisional application claiming the benefit of priority to provisional applications No. 61/588,280 filed Jan. 19, 2012 and No. 61/652,897 filed May 30, 2012, both of which are hereby incorporated by reference in their entireties.

### BACKGROUND OF THE INVENTION

1. Field of the Invention

The present invention relates to methods and apparatus for therapeutically treating Forward Head Posture (FHP), and related disorders of posture, comprising various means for partially obstructing a person's field of vision, said means comprising physical barriers to light according to defined parameters so that said person is compelled to compensate by adopting an adjusted posture, where said adjusted posture is prophylactic for or corrective of a particular disorder related to posture.

2. Description of the Related Art

The following review of related art is intended to provide edifying examples of problems and pitfalls in the design and use of corrective eyewear and treatment of Forward Head Posture and related disorders. The mention of these examples does not constitute an admission that any of the following methods or devices constitute prior art applicable to the present invention. The discussion of the references states what their authors assert, and the applicant reserves the right to challenge the accuracy and pertinency of any of the documents cited herein.

Posture disorders comprise a range of osteopathologies, neuropathies, and other ailments which can create chronic pain and disability. One such disorder is called hyperkyphotic posture or Forward Head Posture (FHP). FHP, which is typically a subconscious and progressive condition, is characterized by the movement of a person's cranial alignment forward and/or downwards such that abnormal strain is placed upon the muscles, ligaments, fascia and bones of the upper body, especially around the neck and shoulders. This condition can become so severe that it impacts vital bodily functions and actually increases mortality rates among affected persons. The forward head carriage that is characteristic of FHP can result not only from genetic and/or age-related degeneration of the affected tissues, but also from repetitive poor head and neck posture. Its increasing prevalence in society is a result of the increasingly wide spread repetitive adoption of forward head postures among today's sedentary population, such as when a person reads at a desk, peers into a handheld device, or works at a computer terminal for extended periods of time on a daily basis. Therefore, both corrective and prophylactic methods and apparatus to treat such conditions are desirable.

Most attempts to correct posture are directed toward the spine, shoulders and pelvis. “Head position takes precedence over all others. The body follows the head. Therefore, the entire body is best aligned by first restoring proper functional alignment to the head.” Cailliet R, Gross L, Rejuvenation Strategy. New York, Doubleday and Co. 1987. “The extra pressure imposed on the neck from poor posture flattens the normal cervical curve resulting in abnormal strain on muscles, ligaments, fascia and bones.” American Journal of Pain Management, January 2008, 4:36-39. Persistent forward head posture increases compressive loads upon the upper thoracic vertebra, and is also associated with the development of Upper Thoracic Hump, which can devolve into Dowager Hump when the vertebra develop compression fractures (anterior wedging). FHP can lead to long term complications such as osteoarthritis. It promotes accelerated aging of intervertebral joints resulting in degenerative joint disease. Posture impacts and modulates all bodily functions from breathing to hormonal production. Back pain, neck pain, headache, mood, blood pressure, pulse and lung capacity are among the many conditions influenced by faulty posture.

Current therapies for treating FHP and other posture disorders focus on corrective surgery, pain management, braces that mechanically force correct posture, and chiropractic rehabilitation. These measures are costly and crude, and they are not effective in dealing with the subconscious nature of postural disorders. In light of the above, there exists a need for a methods and apparatus that both prevent and treat subconscious posture disorders like FHP in real time and in an economical, simplified, convenient, and effective manner.

The present invention is distinguished from those unrelated appliances that block peripheral vision in persons with disorders such as eye sensitivity disorders, which appliances are disclosed in references similar to U.S. Pat. No. 5,402,189 to Gill entitled “Side Shield for Eyeglasses and Method of Making the Same,” issued Mar. 28, 1995. This distinction is manifold, but is primarily because the present invention blocks a portion of the wearer's actual field of vision, as opposed to the peripheral vision, where the peripheral vision is not considered part of the field of vision in this disclosure. Similarly, the present invention is distinguished from those inapplicable appliances which completely occlude an entire field of vision, such as those disclosed in references like U.S. Pat. No. 5,927,279 to Oviatt, entitled “Eye Patch,” issued Jul. 27, 1999, because the present invention derives substantial utility from blocking only a therapeutically or strategically determined portion of the field of vision. The present invention leaves the remainder of the field of vision accessible to the user thereby inducing the user to consciously or subconsciously make a posture adjustment to take advantage of said accessible remainder, which utility could never be elicited in an apparatus that occludes the entirety of a field of vision.

### SUMMARY OF THE INVENTION

The present invention satisfies the above needs. The present invention describes a therapeutic apparatus for occluding a portion of a person's field of vision useful for preventing and treating a range of disorders related to body posture and/or the nervous system, particularly Forward Head Posture (FHP). The present invention also includes methods for manufacturing, adjusting, and using the various embodiments of the apparatus.

The apparatus comprises a means for creating an obstruction in a zone within the field of vision of a person to therapeutically treat said disorders relating to body posture. In a preferred set of embodiments, the obstruction, which can be called an “eyesight blocker” is mounted in or on a lens, or a pair of lenses, such as those within a pair of eyeglasses. In other embodiments, particularly where the person undergoing therapy has no need for corrective eyewear, the means for creating the partial obstruction may be mounted to a wearable frame without any lenses, or with non-vision-corrective translucent or transparent panes instead of lenses. As an alternative to eyeglass frames (i.e., those types of frames which fit over the ears or wrap around the head), the means for mounting or positioning the eyesight blocker may be a small nose-mounted fixture, such as a pair of pads or clips that fit upon the bridge of the nose and suspend the attached eyesight blocker in front of the wearer's eye(s), or any other means known in the art.

Said eyesight blocker, and its means for creating a partial obstruction in a field of view, may comprise a solid material (e.g., a plastic or metal, tab, film, flange, beam, spoke, fabric, disc, etc.), an adhesive film, an electronic panel where polarization or transparency is electrically controlled, or, said means may be integrated as a component aspect of the shape and manufacture of a lens itself, such as where the lens is differentially shaped to create a zone of opacity or special optical features. Typically, the obstruction of eyesight created by the present invention is achieved in substantially the same way for both eyes (e.g., for both lenses in a pair of eyeglasses). However, each obstructing means can also be fashioned to be independently adjustable, such that, for example, the obstructing means and its mode of operation can be independently fashioned and/or independently adjustable for each eye of one person.

Said obstruction(s) is/are created or imparted in a specifically desired or therapeutically determined zone within the field of vision of the wearer such that the obstruction compels the person wearing the apparatus to compensate for it by adjusting their head posture in a desirable way in order to see around the obstruction. For example, regarding a person suffering from or at risk of FHP, the obstruction may occlude an upper portion of the field of vision such that the wearer must tilt his or her head backwards in order to see straight ahead. The precise positioning (e.g., localization, boundaries, distance from the retina) of the obstruction is adjustable to correspond to the degree and characteristics of the posture correction that is desired. The obstruction means may also be variably and situationally adjustable so that it works as intended while the person is both ambulatory and sitting at a desk, for example. In the latter situation, the bottom edge of the occlusion zone would be set to a lower position in the field of vision, in order to achieve the same therapeutic result, than in the former situation, since the eyes inherently focus at more of a downwards angle when a person is sitting at a desk doing work than when walking or standing. This adjustability can be achieved in a variety of ways, including but not limited to: an eyesight blocker comprising folding panels or flaps such that a portion can be folded to expand or contract the effective area of the eyesight blocker; an eyesight blocker comprising detachable panels or parts such that the effective zone of occlusion created by the eyesight blocker can be expanded or contracted by attaching or removing, respectively, said panels and parts; one or more sliding pieces slidably attached to a mounting piece such that the size of the eyesight blocker is effectively enlarged or contracted by sliding said pieces relative to each other; a panel that rolls up like a window shade; a panel comprising dynamic electronics, such as liquid crystal displays, plasma displays, projection displays, or the like (e.g., any means known in the art of wearable computer displays), that can be activated or adjusted under electronic control to modulate the properties of an area in or on the eyesight blocker; and, a series of thin strips removably attached by adhesives, static electricity, or comparable attachment means to a lens, transparent pane, or similar translucent barrier such that the peeling off, cutting up, or shaping of said strips creates variable shapes and sizes of a barrier to light within the field of vision of a person.

The size and position of the obstruction can also be adjusted to induce different changes in posture for different patients with different conditions. For example, a larger obstruction, or one protruding farther into the field of view, will induce a wearer to compensate with a more extreme and persistent adjustment of head posture. Normally, a physician would assist in making such therapeutic determinations. In many cases, the patient may undergo supervised practice and instruction in using the apparatus.

The eyesight blocker may be permanently fixed element in the lens, such as an etching, or it may be optionally removable so that a person undergoing treatment could regain a complete field of vision under circumstances where it may be necessary to do so. For example, in a preferred embodiment the means for creating the obstruction is a clip made of metal, plastic, rubber, a composite of these, or any other suitable material that allows it to be snapped, clipped, slid, or otherwise reversibly attached to an edge of a lens or frame of a pair of eyeglasses. The invention is also amendable to surgically implantable embodiments of the apparatus as well as application in contact lenses, prosthetic devices, and the like.

In embodiments in which the obstruction means is attached to prescription lenses, or in persons requiring corrective lenses, the obstruction means may itself comprise the characteristics of a corrective lens, particularly with respect to the tailored refraction of light. In same or alternative embodiments, the obstruction means may generate said partial obstruction by filtering one or more wavelengths of light from ambient light passing through it or relative to the non-obstructed field of vision. For example, the obstruction zone may comprise a “red colored” zone within the field of vision of the person wearing the apparatus. In yet other embodiments, the obstruction means does not refract, converge, or diverge light passing through it.

In preferred embodiments, the eyesight blocker comprises a means for creating a partial obstruction in the field of vision that is positioned on or within a second material that intervenes in the wearer's field of vision but does not obstruct vision. For example, the eyesight blocker may be attached in or on a transparent lens, screen, or other transparent or translucent material. This is particularly evident in embodiments where the means for creating the obstruction is an etching, and adhesive panel, or a colored region of a lens, for example. In some embodiments, an eyesight blocker comprises a single component part of the apparatus, without any other material to intervene in the wearer's field of vision associated with it.

The apparatus and methods of the present invention may be used similarly both to treat existing posture related conditions and to prevent them in unaffected people. The present invention prevents posture problems by maintaining healthy head, neck, and upper body posture when applied therapeutically to a person engaged in any activity which requires or induces them to adopt an unhealthy or undesirable posture. Because a person will subconsciously adopt an unhealthy or undesirable posture, behavior techniques alone will not work prophylactically to prevent diseases like FHP. The present invention can be applied to preclude a person from adopting forward head carriage postures and other unhealthy or undesirable positions of the body if and when the eyesight blocker is positioned so that tasks or objects in the environment cannot be seen or performed when such an undesirable posture is adopted, because the eyesight blocker is in the way. Instead the person must move the head and body into a desirable position to see through or around the eyesight blocker wherein said desirable position permits them to see and perform the intended activity or object. Thus, the present invention can modify the behavior of a person to discourage or preclude the adoption of certain postures and body positions that are known or suspected to cause posture-related disorders.

In theory, the wearer or user of the present invention can be any animal having both a retina and the cognitive ability to compensate for a partial obstruction in its field of vision by adjusting its posture. The use of the present invention, for example, could be used to correct posture or behavior problems in mammals which are prone to such disorders, or it may be used to reproduce posture related disorders in mammals for experimental purposes, such as to create animal models of human posture disorders. Such animal models can lead to further therapies and treatments for FHP and related ailments in humans which cause great expense and suffering in societies throughout the world.

### DEFINITIONS OF TERMS

The term “body posture” or simply “posture” encompasses any condition or activity involving neuromuscular coordination, muscle tension caused by holding a part of the body in a certain position, a sense of equilibrium or balance, the function of joints, biological mechanisms related to the movable parts of the body, and the like. Body Posture is intended to be construed broadly. “Disorders related to body posture” is likewise intended to be construed broadly, encompassing disorders currently known as well as those not yet discovered.

The term “lens” is used generally to refer to any material that is translucent or transparent and that is fashioned into a barrier to be worn in association with the eye (e.g., over, in, on, or in front of the eye). The term “lens” is intended to include that which is corrective of flawed eyesight (e.g., because the lens refracts light), as well as that which is not corrective of eyesight, such as shading lenses, decorative lenses, or fashionable lenses.

The interchangeable terms “field of vision,” “field of view,” or “window of vision/view” refer generally to the sum total of visual information received by the eyes of a person at a given time. In preferred embodiments, this term relates to the entire field of view achievable by both eyes of a person. However, the term is not necessarily so limited. In other embodiments this term may refer to a portion or segment of a field of view achievable by said person, and it may apply to only one eye or to both eyes simultaneously. The contexts in which these terms are applied will elucidate their particular meanings in various embodiments. A “portion” of a field of vision may relate to a geometrically defined segment or zone within said field of vision and simultaneously or alternatively to an energetically defined segment or zone thereof, such as one or more wavelengths of light or a fraction of the intensity of light. Thus, a “portion” of a field of vision defines, for example, a 3 mm×3 mm square area blocked by an opaque plastic barrier and by a translucent barrier through which only red light passes. The perimeter would be defined as the sum of the lengths and positions of the boundaries of the barrier. In most embodiments the barrier is a single piece with a perimeter defined by its outer edges, but in other embodiments the barrier may have a complicated geometry or a cut-out section in its interior, where the term “perimeter” is still intended to refer to the entire barrier, regardless of the presence of any such additional “inner” boundaries. The terms “block,” “occlude,” and “obstruct” are synonyms that relate to the achievement of these and similar examples. An “eyesight blocker” is any material or method that blocks, occludes, or obstructs at least a portion of a field of vision. To create an obstruction in a field of vision is to block or occlude or obstruct at least a portion of said field of vision.

Parameters have been “therapeutically determined” whenever the geometric and/or energetic parameters required of an eyesight blocker have been determined by a physician, by the application of sound health care advice, by a desire for behavioral modification related to posture control, or by factors based upon a person's biology and/or need for therapy.

In order for eyesight blockers to have reliable behavior modification effects and/or therapeutic value, they must be interposed between a person's retina and incoming ambient light in a substantially stable attachment. A large degree of unpredictability or random drift in the position of the eyesight blocker would be counterproductive. A simple means for achieving the stable emplacement of an eyesight blocker in a person's field of vision comprises eyeglasses. Eyeglasses are substantially stably positioned upon a person's face in front of the eyes by virtue of elements that are fitted to the ears and nose of said person, according to methods and devices well known in the art. Contact lenses are also useful, although they are prone to rotate somewhat in the eye over time unless particular features are imparted to them to keep their orientation stable. Such features can be derived from treatments applied by eye specialists such as surgical implantation, laser treatments, mild adhesives, stitches, and related techniques in the art of vision correction. Any of these examples, as well as any method or apparatus generally, which successfully position the eyesight blocker in a substantially stable position in the field of vision of the wearer can be said to thereby position said eyesight blocker “upon” the wearer. And, “substantially stable position” is not exclusive of also having adjustability. An adjustable mounting means or an adjustable eyesight blocker can be adjusted to adopt a plurality of “substantially stable” positions.

The forgoing summary has outlined some features consistent with the present invention in order that the following detailed description thereof may be better understood, and in order that the present contribution to the art may be better appreciated. The present invention is not limited in its application, details, or components merely to those set forth in the following description and illustrations. The present invention resides not merely in any one of the features set forth in this specification, but also in the particular combination of all of the features and improvements claimed. Methods and devices consistent with the present invention are capable of other embodiments. Also, the phraseology and terminology employed herein are for the purpose of description and should not be regarded as limiting unless explicitly stated as such.

### BRIEF DESCRIPTION OF THE DRAWINGS

FIG. 1 is a perspective view of a first embodiment of the apparatus of the present invention where the eyesight blocker is a slidably adjustable panel.

FIG. 2 is a top view of said first embodiment.

FIG. 3 is a perspective view illustrating the rear surface of the eyesight blocker of said first embodiment.

FIG. 4 is a top view of the eyesight blocker of said first embodiment.

FIG. 5 is a front view of the eyesight blocker of said first embodiment.

FIG. 6 is a rear view of the eyesight blocker of said first embodiment.

FIG. 7 is a side view of the eyesight blocker of said first embodiment.

FIG. 8 is a first perspective view of a portion of the apparatus of said first embodiment illustrating the glider pin of the slidably adjustable eyesight blocker assembly.

FIG. 9 is a second perspective view of a portion of the apparatus of said first embodiment illustrating the attachment points for said glider pin of the slidably adjustable eyesight blocker assembly.

FIG. 10 is a perspective view of the glider pin of the slidably adjustable eyesight blocker assembly.

FIG. 11 is a cross-sectional view of the glider pin of the slidably adjustable eyesight blocker assembly.

FIG. 12 is a top view of the glider pin of the slidably adjustable eyesight blocker assembly.

FIG. 13 is a top view of a portion of said first embodiment illustrating the position of glider pin assemblies.

FIG. 14 is a perspective view of a person using said first embodiment to regulate posture during the activity of working at a computer station and to prevent and treat Forward Head Posture.

FIG. 15 is a perspective view of a second embodiment of the apparatus of the present invention where the eyesight blocker comprises two independently adjustable means for occluding a portion of a field of vision.

FIG. 16 is a perspective view of a third embodiment of the apparatus of the present invention where the eyesight blocker comprises two independently adjustable means for occluding a portion of a field of vision.

FIG. 17 is a perspective view of a fourth embodiment of the apparatus of the present invention where the eyesight blocker comprises removable and re-attachable means for occluding a portion of a field of vision.

FIG. 18 is a perspective view of a fifth embodiment of the apparatus of the present invention where the eyesight blocker comprises two independently adjustable means for occluding a portion of a field of vision.

FIG. 19 is a perspective view of a sixth embodiment of the apparatus of the present invention where the eyesight blocker comprises an adjustable means for occluding a portion of a field of vision resembling a windowshade.

FIG. 20 is a perspective view of a seventh embodiment of the apparatus of the present invention where the eyesight blocker comprises two pieces of film adhesively attached to the lenses of eyeglasses.

FIG. 21 is a perspective view of the seventh embodiment illustrating that said film may be adjustable by virtue of comprising multiple layers or independently-removable strips attached to the front surface of said lenses.

FIG. 22 is a perspective view of the seventh embodiment illustrating that said film may be adjustable by virtue of comprising multiple layers or independently-removable strips attached to the rear surface of said lenses.

FIG. 23 is a perspective view of an eighth embodiment of the apparatus of the present invention where the eyesight blocker comprises portions of lenses which are fashioned to filter a certain wavelength of light passing through them.

### DETAILED DESCRIPTION OF THE DRAWINGS

Throughout all the Figures, same or corresponding elements are indicated by the same reference numerals.

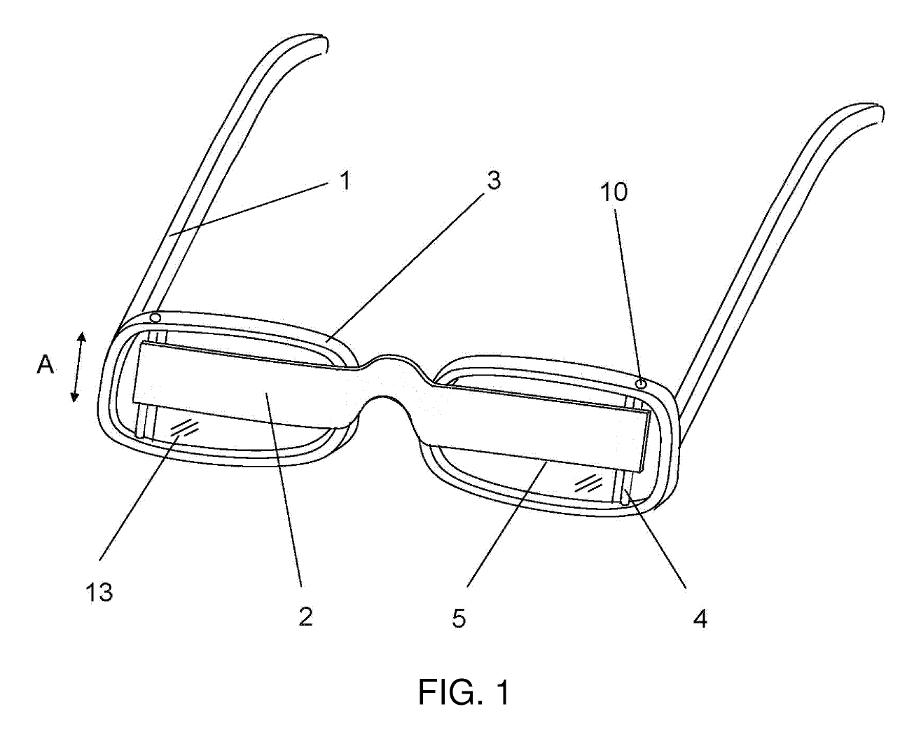

FIG. 1 is a perspective view of a first embodiment of the apparatus of the present invention where the eyesight blocker is a slidably adjustable panel. The eyesight blocker 2 is associated with an eyeglasses frame 3 via a pair of parallel glider pins 4. Said glider pins 4 are securely and vertically associated with eyeglasses frame 3. The height of the lower edge 5 of eyesight blocker 2 can be adjusted by gliding said blocker 2 along the glider pins 4 vertically, as shown by arrows A. Said eyeglasses frame 3 may or may not have lenses in the panes of the eyegalsees. Said eyesight blocker 2 may have various degrees of optical transparency, translucency or various colors. The height of lower edge 5 of eyesight blocker 2 may differ for the left and right portions of eyeglasses frame 3.

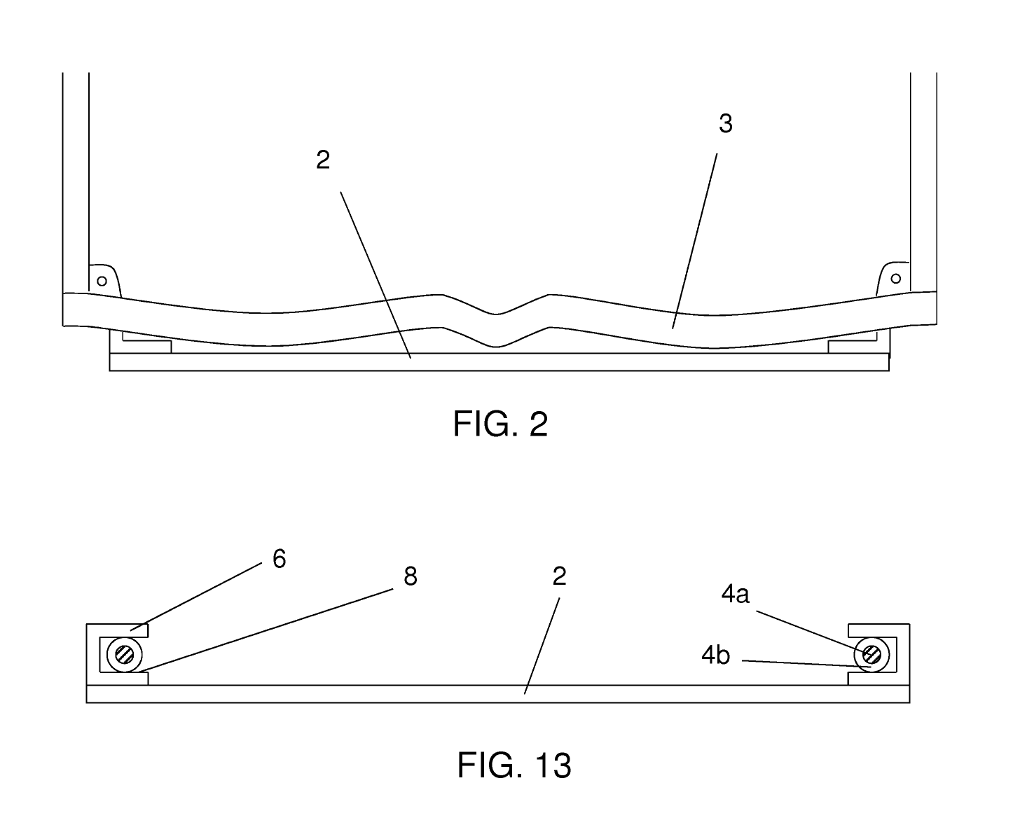

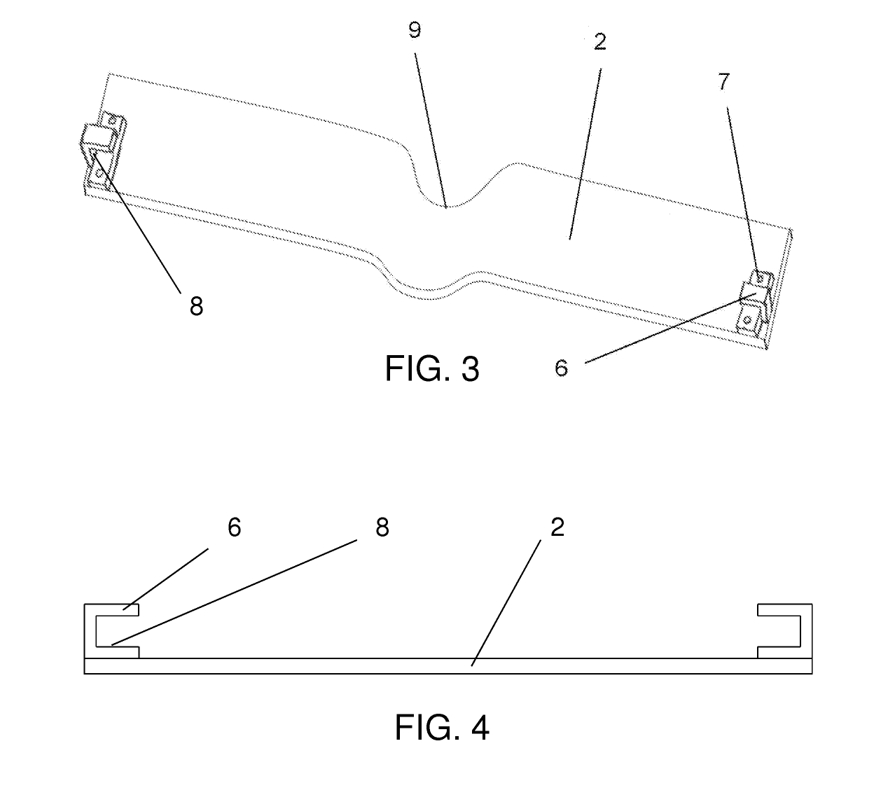

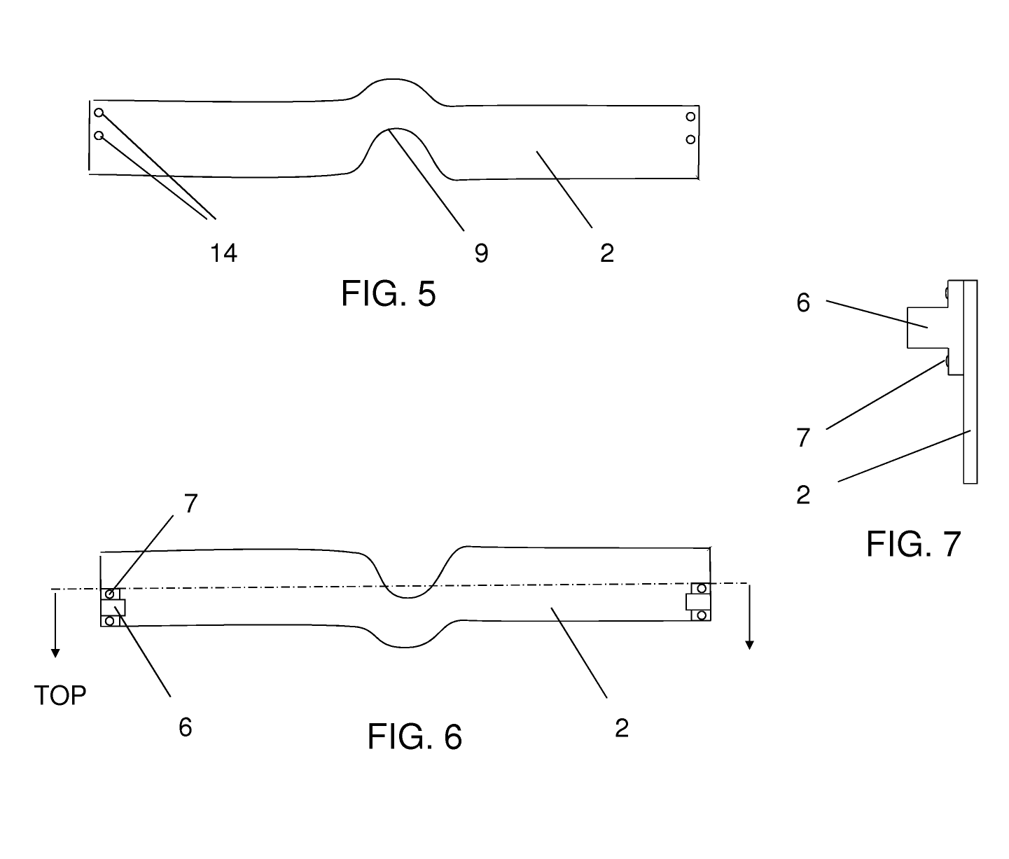

FIG. 2 is a top view of said first embodiment. Eyesight blocker 2 is associated with the eyeglasses frame 3 by being in front of the eyeglasses frame 3 and substantially in parallel to the façade of said frame 3. FIG. 3 is a perspective view illustrating the rear surface of the eyesight blocker of said first embodiment. The eyesight blocker 2 is made of light weight sheet material that is sufficiently less transparent, sufficiently opaque or sufficiently strongly colored. One pair of holders 6 is affixed to said eyesight blocker 2 via a multiple of screws 7. Said pair of holders 6 are mounted on the top portion of said eyesight blocker 2 substantially at the same horizontal level. The gliding channels 8 of said holders 6 are substantially perpendicular to the horizontal level of the eyesight blocker 2. FIG. 4 is a top view of the eyesight blocker of said first embodiment. One pair of holders 6 is affixed to said eyesight blocker 2. FIG. 5 is a front view of the eyesight blocker of said first embodiment. The eyesight blocker 2 is shaped with a curve 9 to enhance its performance of vertical adjustment—to be able to move sufficiently downwards without interfered by user's nose. Holes 14 are prepared for mounting holders 6 onto the eyesight blocker 2 via screws 7. FIG. 6 is a rear view of the eyesight blocker 2 of said first embodiment. One pair of holders 6 is affixed to said eyesight blocker 2 via a multiple of screws 7. Said pair of holders 6 are mounted towards the top portion of said eyesight blocker 2 substantially at the same horizontal level. FIG. 7 is a side view of the eyesight blocker of said first embodiment. One pair of holders 6 is affixed to said eyesight blocker 2 via a plurality of screws 7. Said pair of holders 6 are mounted on the top portion of said eyesight blocker 2 substantially at the same horizontal level.

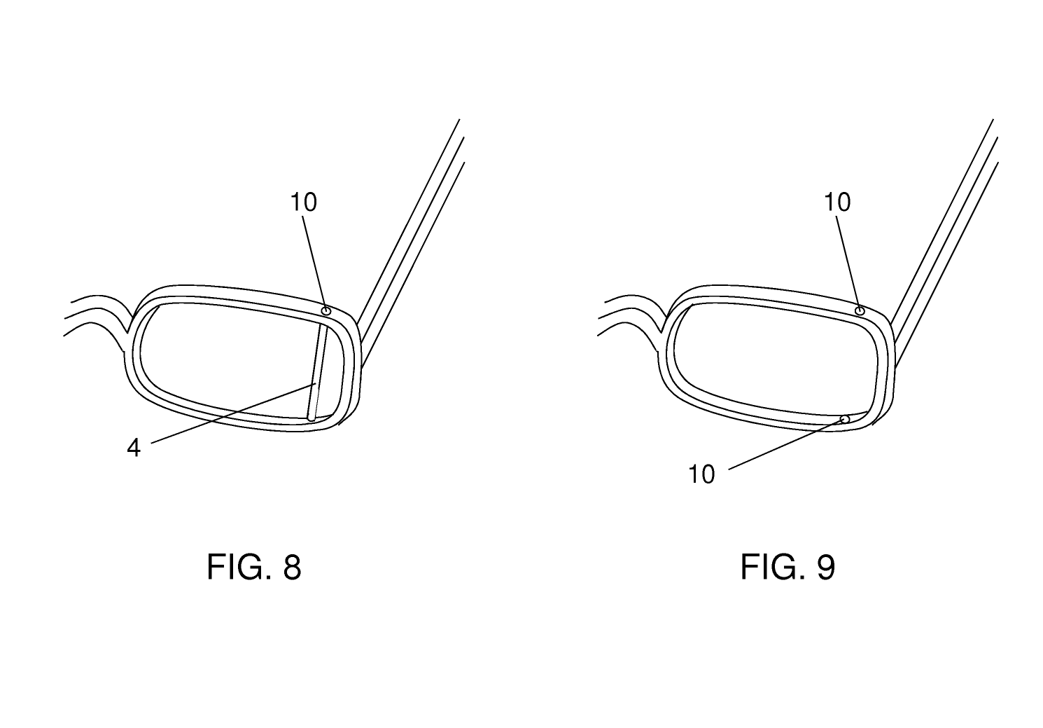

FIG. 8 is a first perspective view of a portion of the apparatus of said first embodiment illustrating the glider pin of the slidably adjustable eyesight blocker assembly. Said glider pin 4 is securely associated with left hand portion of the eyeglasses frame 3, by penetrating said frame 3 via hole 10. In the same manner, a glider pin 4 is securely associated with right hand portion of the eyeglasses frame 3. FIG. 9 is a second perspective view of a portion of the apparatus of said first embodiment illustrating the attachment points for said glider pin of the slidably adjustable eyesight blocker assembly. Holes 10 are prepared on the left hand portion of eyeglasses frame 3 substantially towards the left end of left portion of the eyeglasses frame for securing glider pin 4. In the same manner, corresponding holes 10 are prepared on the right hand portion of eyeglasses frame 3 for securing glider pin 4. Note that other mechanical means for adjusting the position of the eyesight blocker can be applied to the apparatus besides this gliding pin mechanism. A motorized track system or other means to achieve equivalent results are contemplated herein.

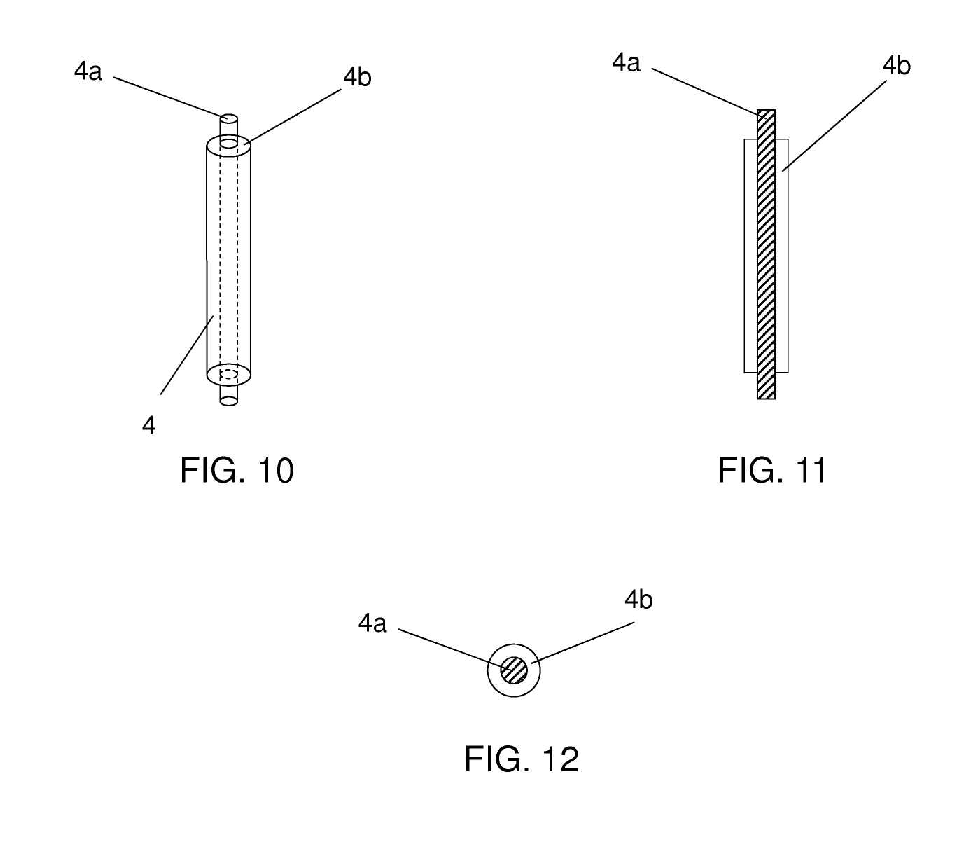

FIG. 10 is a perspective view of the glider pin of the slidably adjustable eyesight blocker assembly. The glider pin 4 has a cylindrical structure, with a cylindrical pin 4a made of a stiff material that is surrounded by a cylindrical cover 4b made of a soft rubbery material. The stiff material may be a metal, an alloy, a hard and durable polymer. The stiffness of the material is to assure the structural integrity of the glider pins 4 and the gliding performance of the eyesight blocker 2 along the glider pins 4. The rubbery material of 4b is to better facilitate the gliding channels 8 to hold onto the glider pin 4 snugly to allow reasonably easy gliding of the eyesight blocker 2 along the glider pins 4 vertically, but will be able to hold the eyesight blocker 2 in its vertical position reasonably securely once in desired vertical position. FIG. 11 is a cross-sectional view of the glider pin of the slidably adjustable eyesight blocker assembly. The glider pin 4 has a cylindrical structure, with a cylindrical pin 4a made of a stiff material that is surrounded by a cylindrical cover 4b made of a soft rubbery material. FIG. 12 is a top view of the glider pin of the slidably adjustable eyesight blocker assembly. The glider pin 4 has a cylindrical structure, with a cylindrical pin 4a made of a stiff material that is surrounded by a cylindrical cover 4b made of a soft rubbery material. FIG. 13 is a top view of a portion of said first embodiment illustrating the position of glider pin assemblies. Eyesight blocker 2 holds on the glider pins 4 via holders 6. The opening of the gliding channels 8 is small enough to hold onto the glider pin 4 snugly to allow reasonably easy gliding of the eyesight blocker 2 along the glider pins 4 vertically, but will hold the eyesight blocker in its vertical position reasonably securely once in desired vertical position. Said gliding channels 8 of holders 6 may be coated with rubbery material to enhance and assure such performance. The rubbery coating of gliding channel 8 and rubbery cover 4b of glider pin 4a assure the performance of allowing reasonably easy up and down gliding of the eyesight blocker 2 along the glider pins 4, while holding the eyesight blocker 2 in its vertical position reasonably securely once in the desired vertical position.

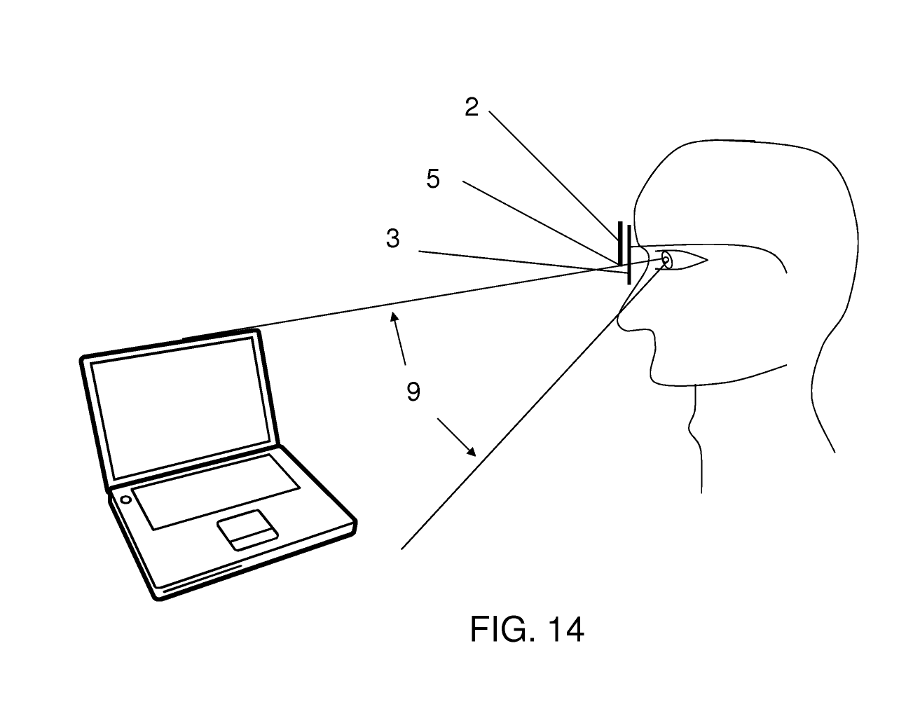

FIG. 14 is a perspective view of a person using said first embodiment to regulate posture during the activity of working at a computer station and to prevent and treat Forward Head Posture. Eyesight blocker 2 interferes or obstructs the upper or upper plus upper-middle portions of field of user's vision. A user's eyesight underneath the eyesight blocker 2 is not interfered or blocked by the eyesight blocker 2. A user will realize that her upward- or upward-plus horizontal-looking eyesight is completely or partially interfered or blocked by the eyesight blocker 2, when the lower edge 5 of eyesight blocker 2 is set sufficiently low in relation to eyeglasses frame 3, and when her head or neck tilts forward or her back slouches. A user will only be able to see her objective comfortably through the visual area 9 available underneath the eyesight blocker 2. This will force the user to keep up her head in order to see her objective, hence encourage an upright head posture to reduce and prevent Forward Head Posture, and mitigate the health issues caused by Forward Head Posture. Within an effective range of adjustment, the lower the lower edge 5 of eyesight blocker 2 is set, the more the user will need to tilt her head backward to see her objective. By setting the appropriate vertical position of lower edge 5 of eyesight blocker 2, user will be able to see her objective comfortably while automatically maintaining a healthy upright posture at work, at home or on the road, without the need of external reminder or harsh correction, hence successfully reducing and preventing Forward Head Posture.

Research using the apparatus of the present invention has shown that placing the lower edge 5 of eyesight blocker 2 in front of computer user's eyes at about the height of her pupil causes her eyesight beam to tilt downwards and reduces the forward tilt of her head from approximately 25-30 degree to 0-5 degree, measured from forward leaning angle of the façade of her face, depending on the individual. Such reduction of forward tilt of head substantially eliminates forward head posture. Proper adjustment of the position of area of partial obstruction of vision, or of the eyesight blocker 2, is critical to achieve desired therapeutic outcome. Even a millimeter of deference in the positioning of the area of obstruction of vision may separate the success or failure of the corrective or preventative therapy.

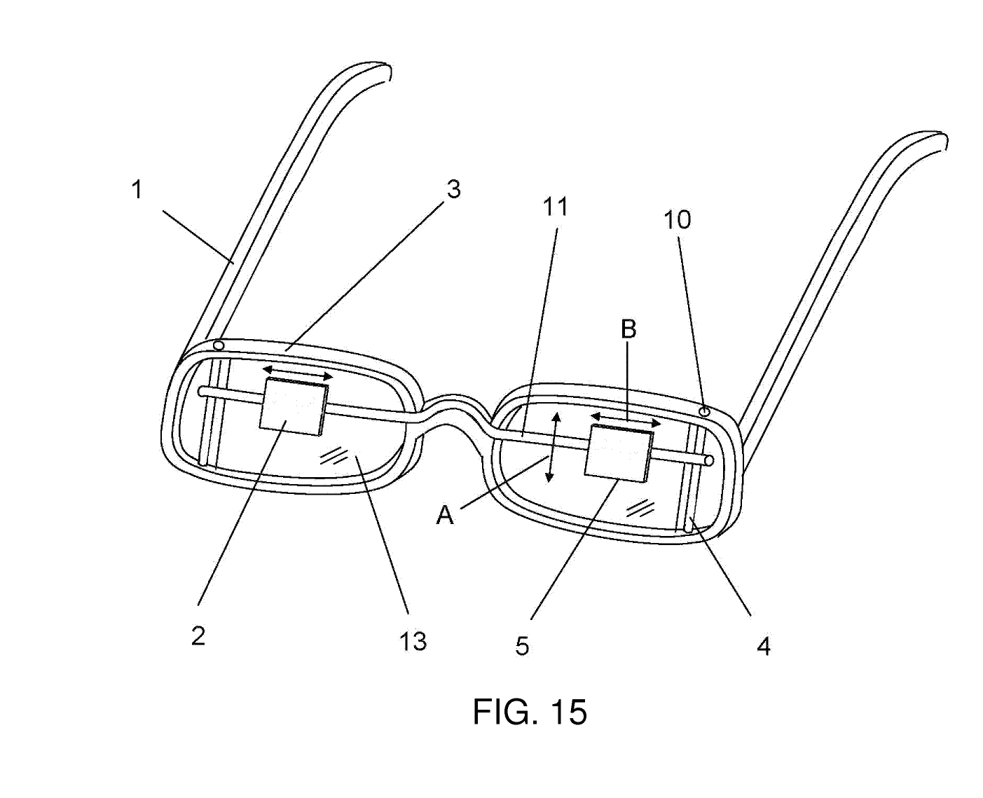

FIG. 15 is a perspective view of a second embodiment of the apparatus of the present invention where the eyesight blocker comprises two independently adjustable means for occluding a portion of a field of vision. Two eyesight blockers 2 are associated with an eyeglasses frame 3 via horizontal bar 11. Said horizontal bar 11 can glide up and down along the glider pins 4 vertically for adjusting the vertical position of the eyesight blockers 2, as shown by arrows A. The horizontal position of said two eyesight blockers can be separately adjusted to be aligned with user's two eyes, as shown by arrows B.

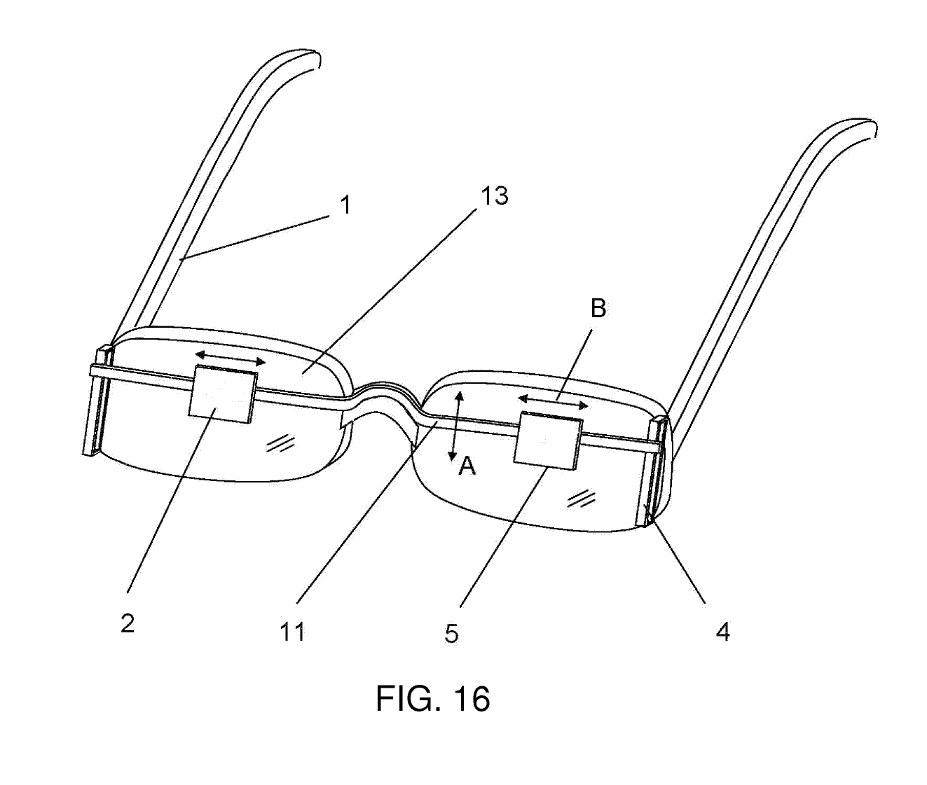

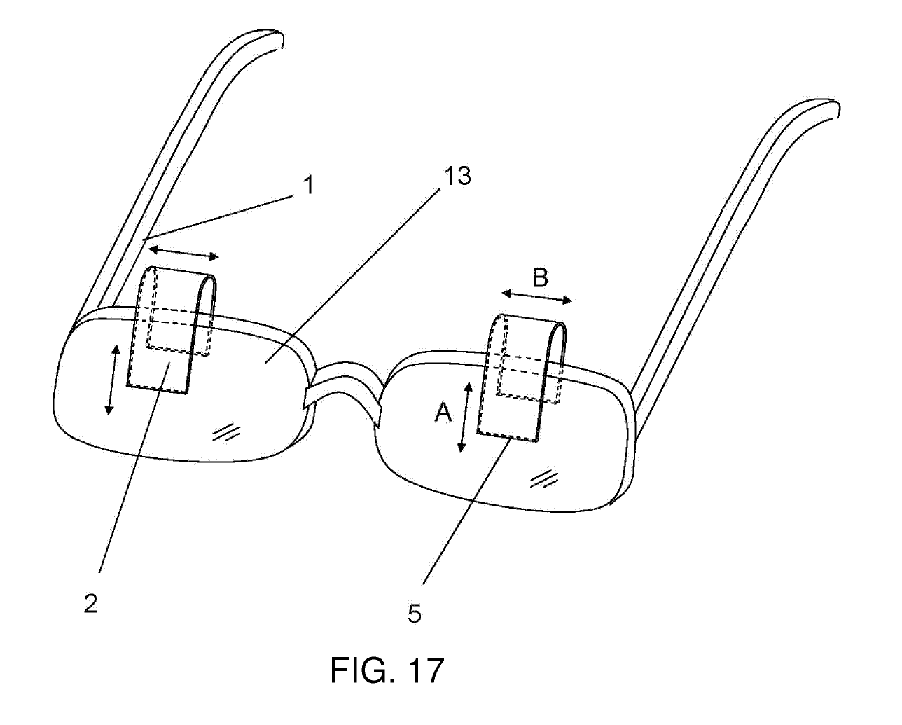

FIG. 16 is a perspective view of a third embodiment of the apparatus of the present invention where the eyesight blocker comprises two independently adjustable means for occluding a portion of a field of vision. There is no eyeglasses frame in this embodiment. Two eyesight blockers 2 are associated with lenses 13 via horizontal bar 11 and vertical gliders 4. Said horizontal bar 11 can glide up and down along the gliders 4 vertically for adjusting the vertical position of the eyesight blockers 2, as shown by arrows A. Said gliders 4 are fixed onto the lenses 13 at the both ends of the eyeglasses. The horizontal position of said two eyesight blockers can be separately adjusted to be aligned with user's two eyes, as shown by arrows B.

FIG. 17 is a perspective view of a fourth embodiment of the apparatus of the present invention where the eyesight blocker comprises removable and re-attachable means for occluding a portion of a field of vision. Two separate eyesight blockers 2 are made of substantially flexible clips, and are clipped onto two lenses 13. Said clips may be made of metal or polymer materials, and is sufficiently sturdy yet flexible, and are capable of securely holding onto the lenses 13 at desired vertical position while being able to move up and down to adjust the vertical position of the lower edges 5 of the eyesight blockers 2, as shown by arrows A. The horizontal position of said two eyesight blockers can be separately adjusted to be aligned with user's two eyes, as shown by arrows B. The contact surface of the clips 2 with the lenses 13 may be associated with a coating, such as a layer of soft rubbery material to enhance the performance of proper gliding of the clips on the lenses both vertically and horizontally, but also the performance of holding the clips 2 at the desired vertical and horizontal locations.

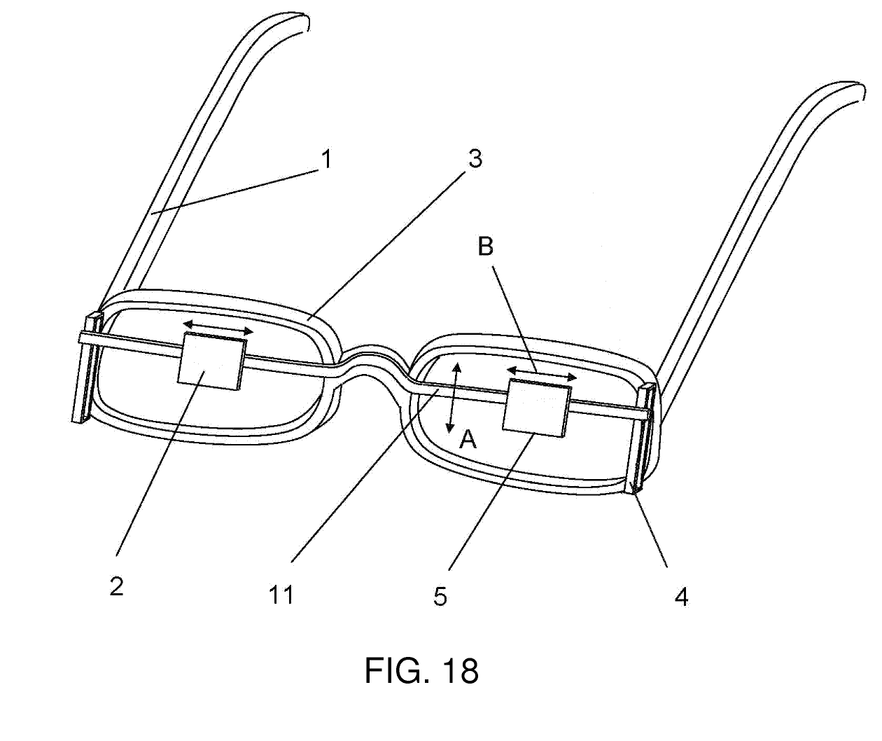

FIG. 18 is a perspective view of a fifth embodiment of the apparatus of the present invention where the eyesight blocker comprises two independently adjustable means for occluding a portion of a field of vision. Two eyesight blockers 2 are associated with an eyeglasses frame 3 via horizontal bar 11. Said horizontal bar 11 can glide up and down along the gliders 4 vertically for adjusting the vertical position of the eyesight blockers 2, as shown by arrows A. The horizontal position of said two eyesight blockers can be separately adjusted to be aligned with user's two eyes, as shown by arrows B. This assembly is void of any lenses.

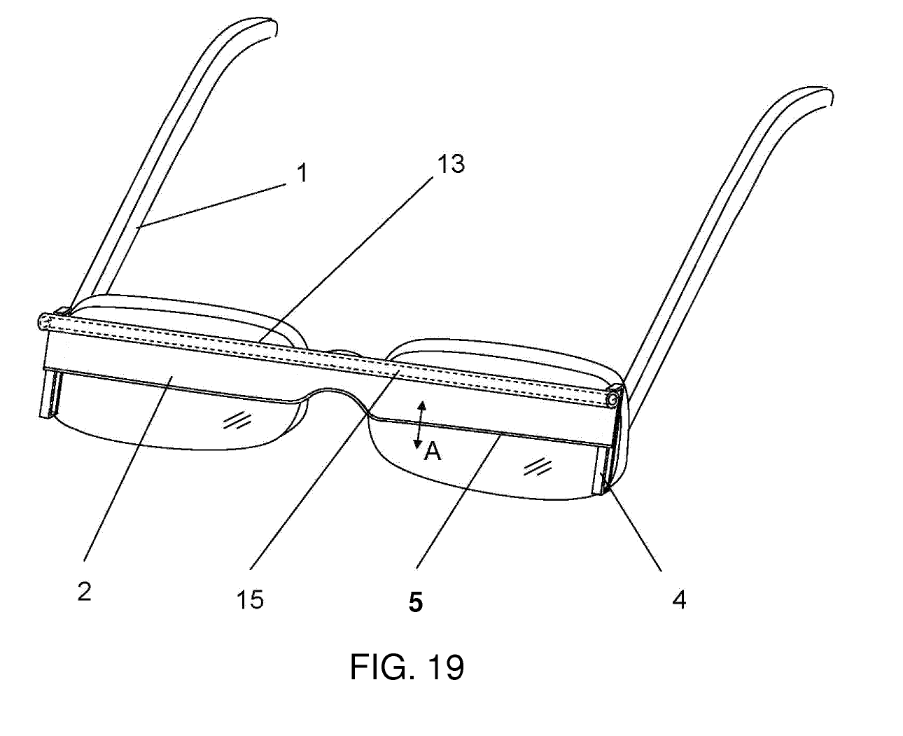

FIG. 19 is a perspective view of a sixth embodiment of the apparatus of the present invention where the eyesight blocker comprises an adjustable means for occluding a portion of a field of vision. Eyesight blocker 2 is a window-curtain- or shade-like component and is associated with lenses 13 via vertical gliders 4. Said window-curtain like eyesight blocker 2 is rolled and stored on rolling bar 15 (e.g., a dowel). Rolling bar 15 is affixed on gliders 4. The lower end 5 of curtain like eyesight blocker 2 can be pulled down or pushed up along glider 4, as shown by arrows A. Said gliders 4 are fixed onto the lenses 13 at both ends of the eyeglasses.

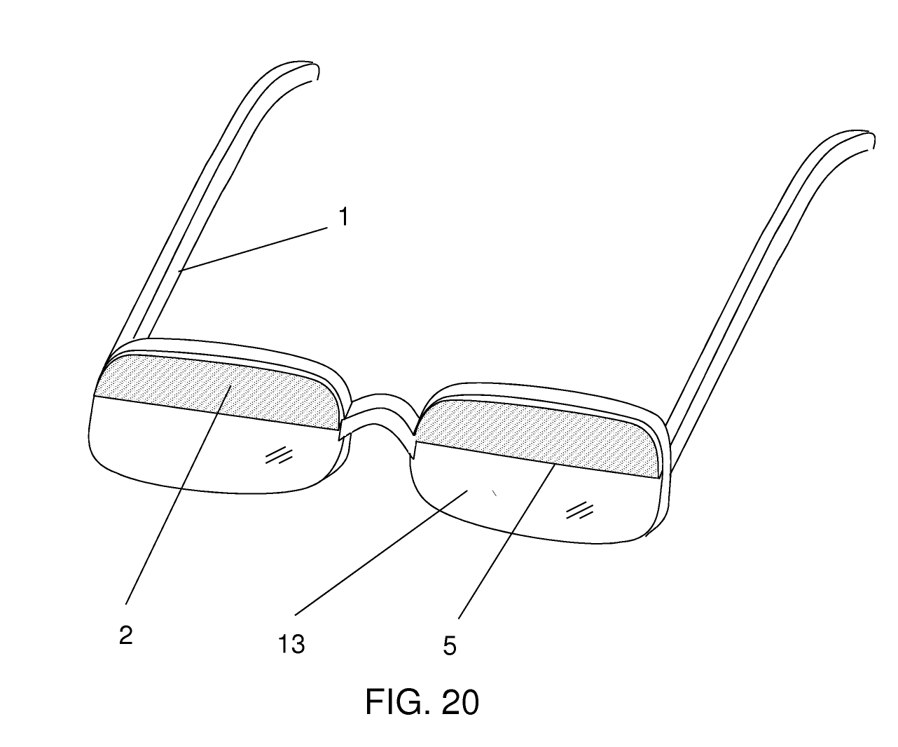

FIG. 20 is a perspective view of a seventh embodiment of the apparatus of the present invention where the eyesight blocker assembly comprises two pieces of film adhesively attached to the lenses of eyeglasses. Eyesight blocker 2 is a piece of film and is attached to the front side of lenses 13 via methods such as, but not limited to, adhesive gluing. The lower edge 5 of eyesight blocker 2 is virtually fixed unless user chooses to detach and reattach said eyesight blocker 2 to lens 13. Said eyesight blocker substantially obstructs the upper or upper plus upper middle portions of user's vision fields, and leave lower, lower plus middle, or lower plus lower-middle portions of user's vision field unobstructed. The color, transparency or translucency of at least one portion of said eyesight blocker is visually differentiable from those of said lens or ambient air in normal living environment. The addition of said film may interfere with, distort or block the upper or upper plus upper-middle portions of field of user's vision, hence to encourage the user to tilt back her head and maintain a healthy upright posture. In a variation of this embodiment, the eyesight blocker 2 may be a polarizable material or panel, and it may also comprise an electronically or interactively polarizable panel whose properties (including size, shape, transparency, translucency, etc.) may be electronically controlled to accommodate various uses.

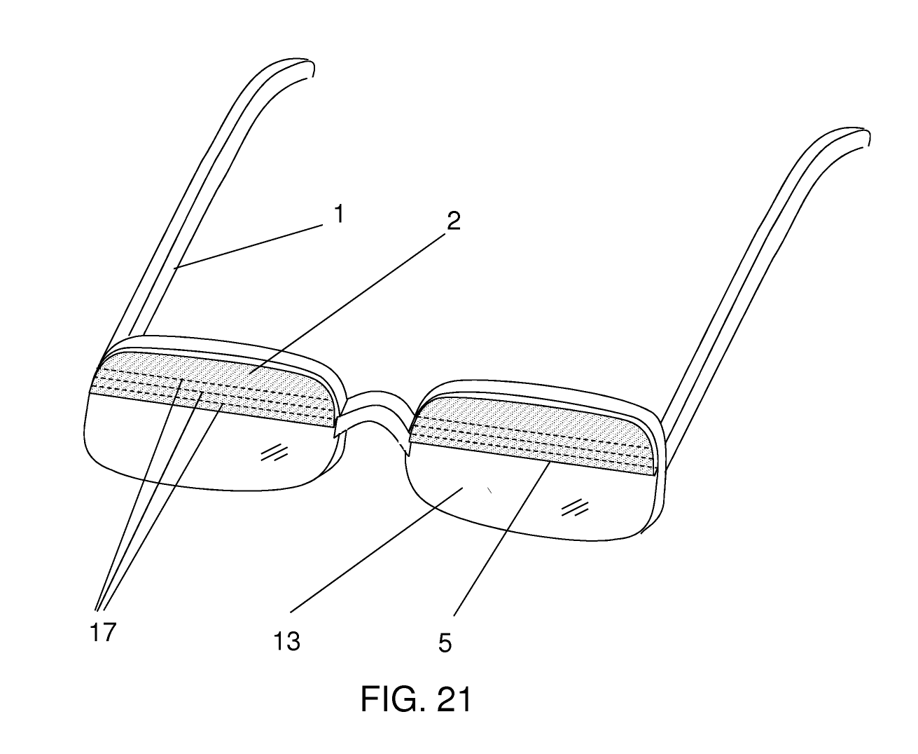

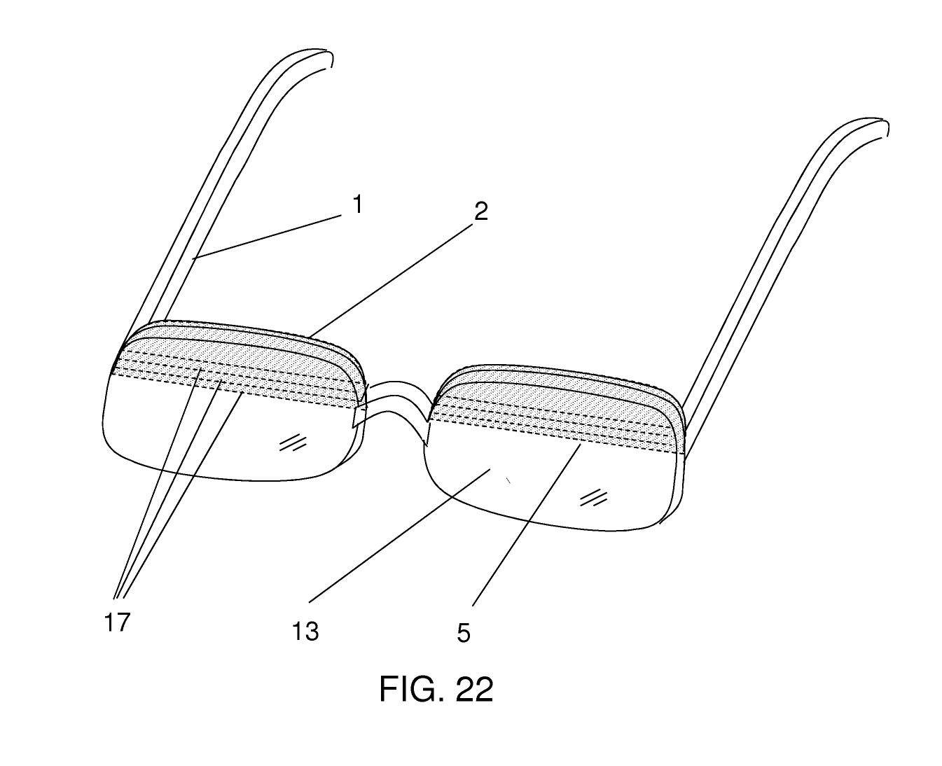

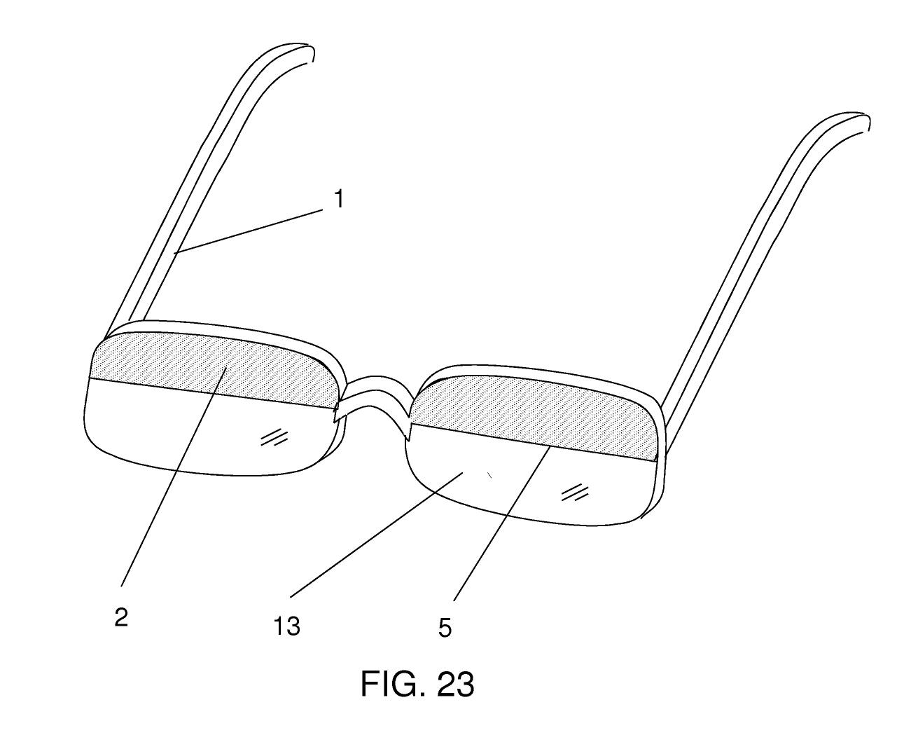

FIG. 21 is a perspective view of the seventh embodiment illustrating that said film may be adjustable by virtue of comprising multiple layers or independently-removable strips attached to the front surface of said lenses. In one version of this embodiment, the lower portion of said eyesight blocker is pre-sliced into a multiple of parallel strips 17 and can be peeled off of lens 13 to move the lower edge of said eyesight blocker slightly upwards and to increase user's window of vision slightly upwards. A multiple of parallel strips allows a multiple of choices of the vertical position of said eyesight blocker 2's lower edge 5. FIG. 22 is a perspective view of the seventh embodiment illustrating that said film may be adjustable by virtue of comprising multiple layers or independently-removable strips attached to the rear surface of said lenses, wherein the eyesight blocker 2 with its lower portion pre-sliced into a multiple of parallel strips 17 is applied to lens 13 from behind.

FIG. 23 is a perspective view of an eighth embodiment of the apparatus of the present invention where the eyesight blocker comprises portions of lenses which are fashioned to filter a certain wavelength of light passing through them. Eyesight blocker 2 is an area (of lens 13) where color pigment is integrated in or applied. The addition of said color pigment may interfere with, distort or block the upper or upper plus upper-middle portions of field of user's vision, hence to encourage the user to tilt back her head and maintain a healthy upright posture. Instead of pigment, any type of mechanical etching, resurfacing, coating, or other mechanical alteration of the transparency of a material can be applied to generate a means for creating a partial obstruction in the field of vision of a person using the apparatus.

### Sample of Operation

In preferred embodiments of the present invention, the apparatus works similarly to a pair of eyeglasses, in that a user would simply wear it as a pair of normal eyeglasses like those shown in FIG. 14. The user, or the user's physician, has the ability to adjust the vertical position of the lower edge of the eyesight blocker to best suit her needs, in case the eyesight blocker's lower edge in many embodiments. The user would maintain the apparatus upon the eyes and then go about living and working as in normal daily life as if wearing a normal pair of sunglasses. However, the eyesight blocker will discourage or impair the user's ability to perform normal activities while in an unhealthy posture. FIG. 14 provides an sample of what a user would experience while working at a desk where focusing upon a computer terminal, for example. Eyesight blocker 2 interferes with and/or obstructs the upper or upper plus upper-middle portions of field of user's vision. However, the user's ability to see from the portion of the view that is not occluded by the eyesight blocker is available to provide a clear line of sight, but only when the user tilts the head backwards enough to maintain the therapeutically desired head posture and/or body posture. A user will realize that her upward- or upward-plus horizontal-looking eyesight is completely or partially interfered or blocked by the eyesight blocker 2, when the lower edge 5 of eyesight blocker 2 is set sufficiently low in relation to eyeglasses frame 3, and when her head or neck tilts forward or her back slouches. A user will only be able to see her objective comfortably through the visual area 9 available underneath the eyesight blocker 2. This will compel the user to keep up her head in order to see her objective, hence encourage an upright head posture to reduce and prevent Forward Head Posture, and mitigate the health issues caused by Forward Head Posture. The lower the lower edge 5 of eyesight blocker 2 is set, the more the user will need to tilt her head backward to see her objective. By setting the right vertical position of lower edge 5 of eyesight blocker 2, user will be able to see her objective comfortably while automatically maintaining a healthy upright posture at work, at home or on the road, without the need of external reminder or harsh correction, hence successfully reduce and prevent Forward Head Posture.

The perimeter of the eyesight blocker can be adjusted in multiple ways to achieve optimum results in different patients or during different activities. A first type of adjustability is denoted by the ability to slide the entire eyesight blocker up and down along gliding pins, where the perimeter of the eyesight blocker is adjusted not with respect to its dimensions but only with respect to its position in space. A second type of adjustability is denoted by the ability to deform or reshape the eyesight blocker such that its dimensions change, as in the embodiment in which the eyesight blocker resembles a window-shade. A third type of adjustability is inherent in embodiments where the means for creating a partial obstruction comprise multiple pieces, such as strips of an adhesive film, that can be layered upon or peeled off of the eyesight blocker to expand or contract the perimeter of the eyesight blocker, for example. It is a useful feature of the present invention that the area occupied by the eyesight blocker can be adjusted in its size, its shape or its position in space, according to various therapeutic requirements, and according to different embodiments of the apparatus.

It should be emphasized that the above described embodiments of the present invention exemplify some, but not all, possible implementations of the present invention and have been set forth in order to provide a clear understanding of its qualities. Those skilled in the art will appreciate that the conception upon which this disclosure is based may readily be utilized as a basis for designing of other structures, methods, and systems for carrying out the several purposes of the present invention. The following claims should be regarded as encompassing equivalent and various constructions insofar as they do not depart from the spirit and scope of the methods and devices consistent with the present invention.

I claim:

1. A therapeutic apparatus for occluding a portion of a person's field of vision, useful for the treatment of disorders relating to body posture, the apparatus comprising:
at least one eyesight blocker, said eyesight blocker comprising a means for creating a partial obstruction in the field of vision of a person,
said partial obstruction comprising a perimeter that circumscribes a therapeutically determined zone within said field of vision,
a means for mounting said eyesight blocker in a substantially stable position upon said person,
such that said partial obstruction requires said person to compensate for said partial obstruction by making an adjustment in body posture,
wherein said adjustment in body posture is corrective of a disorder relating to body posture.

2. The apparatus of claim 1, wherein the disorder is Forward Head Posture.

3. The apparatus of claim 1, wherein the means for creating a partial obstruction in the field of vision of a person is opaque.

4. The apparatus of claim 1, wherein said eyesight blocker further comprises a lens.

5. The apparatus of claim 1, wherein the means for mounting said eyesight blocker in a substantially stable position upon said person comprises an eyeglasses frame.

6. The apparatus of claim 1, wherein the means for mounting said eyesight blocker is adjustable, such that the area occupied by said partial obstruction within said field of vision may be therapeutically adjusted from a first substantially stable position upon said person to a plurality of another substantially stable position.

7. The apparatus of claim 1, further comprising gliding pins attached to said means for mounting said eyesight blocker, and one or more corresponding holders on the eyesight blocker for receiving said gliding pins, whereby said eyesight blocker is slidably adjustable.

8. The apparatus of claim 7, wherein said eyesight blocker further comprises a bar transverse to said sliding pins upon which are attached two independently adjustable panels, each of said panels comprising a means for creating a partial obstruction in the field of vision of a person.

9. The apparatus of claim 8, wherein said independently adjustable panels are slideably attached to said bar.

10. The apparatus of claim 1, wherein the eyesight blocker is removable from and replaceable in the apparatus.

11. The apparatus of claim 1, wherein the eyesight blocker comprises a flexible, generally u-shaped clip by which it firmly attaches around an edge of a pair of eyeglasses at adjustable positions along the perimeter of said pair of eyeglasses.

12. The clip of claim 11, further comprising a coating applied to said clip to modify the frictional properties of said clip and to thereby facilitate attachment and re-attachment of said clip to said pair of eyeglasses.

13. The apparatus of claim 1, wherein the means for creating a partial obstruction in the field of vision of a person is translucent.

14. The apparatus of claim 1, wherein said means for creating a partial obstruction comprises a material that filters one or more particular wavelengths of light.

15. The apparatus of claim 1, wherein said means for creating a partial obstruction comprises an etching.

16. The apparatus of claim 1, wherein the eyesight blocker comprises a curtain-like flexible panel wound around a dowel, where said dowel rotates on its axis to raise and lower said curtain-like flexible panel.

17. The apparatus of claim 1, wherein the means for creating a partial obstruction in the field of vision of a person is dynamically transparent, such that it may alternate between different states of translucency.

18. The apparatus of claim 17, wherein said means is an electronically polarizable panel.

19. The apparatus of claim 1, wherein the means for creating a partial obstruction in the field of vision of a person is adhesively attached to the apparatus.

20. The apparatus of claim 1, wherein the means for creating a partial obstruction in the field of vision of a person is a strip of film attached to a lens.

21. A therapeutic method of preventing and treating one or more disorders relating to body posture, the method comprising:
(a) providing the apparatus of claim 1 comprising one or more of an eyesight blocker, said eyesight blocker comprising a means for creating a partial obstruction in the field of vision of a person, said person suffering from a disorder relating to body posture;
(b) positioning, when worn upon said person, said eyesight blocker such that said partial obstruction obstructs the field of vision such that said person is compelled to adopt an adjusted body posture in order to compensate for said partial obstruction, whereby said adjusted body posture is corrective of said disorder;
(c) thereby preventing and treating said disorder relating to body posture.

22. The method of claim 21, further comprising the step of instructing said person to consistently wear said apparatus during one or more of a particular activity.

23. The method of claim 21, further comprising the step of subjecting said person to a regiment of training whereby said person repetitiously performs one or more of a particular activity while wearing said apparatus under the supervision of a health care professional.

24. The method of claim 21, wherein the disorder is Forward Head Posture.

25. A method for regulating body posture in animals including humans comprising the steps of:
(a) providing the apparatus of claim 1;
(b) mounting said apparatus upon said animal in a substantially stable position such that a portion of the field of vision of said animal is occluded;
(c) adjusting said at least one eyesight blocker so that said portion of the field of vision corresponds to one or more desired geometric and energetic parameters for inducing said animal to adopt one or more corresponding desired body postures while said animal engages in one or more particular activities;
(d) monitoring the body posture of said animal while and after said animal engages in said particular activities;
(e) repeating steps (c) and (d) until said animal is trained to adopt one or more desired body posture during said particular activities;
(f) thereby regulating body posture in said animal.
